(19) United States
(12) Patent Application Publication
(10) Pub. No.: US 2022/0241378 A1
(43) Pub. Date: Aug. 4, 2022

(54) COMPOSITIONS AND METHODS FOR TREATMENT OF CHRONIC GRANULOMATOUS DISEASE

(71) Applicant: RegeneRx Biopharmaceuticals, Inc., Rockville, MD (US)

(72) Inventors: Luigina Romani, Perugia (IT); Enrico Garaci, Rome (IT); Allan L. Goldstein, Reedville, VA (US)

(73) Assignee: RegeneRx Biopharmaceuticals, Inc., Rockville, MD (US)

(21) Appl. No.: 17/610,361

(22) PCT Filed: May 12, 2020
(86) PCT No.: PCT/US2020/032471

§ 371 Date: Nov. 10, 2021

Related U.S. Application Data
(60) Provisional application No. 62/848,118, filed on May 15, 2019.
(60) Provisional application No. 62/934,149, filed on Nov. 12, 2019.

(51) Int. Cl. A61K 38/22; A61P 13/06; A61P 21/02

(52) U.S. Cl. CPC A61K 38/2292; A61P 21/02; A61P 13/06

(57) ABSTRACT

Methods, compositions and kits for treating autophagy mediated diseases and disorders are disclosed as well as methods of treating a subject suffering from chronic granulomatous disease (CGD) by administering an effective amount of a thymosin polypeptide or variant thereof to the subject.

### FIELD OF THE DISCLOSURE

This disclosure relates to compositions and methods for inhibiting, treating or reducing the likelihood of the onset of an autophagy-mediated disease in a subject.

### BACKGROUND

Autophagy is the process mediating lysosomal degradation of target materials in the cell to maintain cellular homeostasis. Autophagy is critical for sensing microorganisms or their metabolic products by translating the signaling host physiological responses at mucosal surfaces. Thus, autophagy may play a crucial role in maintaining intestinal homeostasis. Genetic studies of inflammatory bowel diseases (IBD) have revealed important roles for autophagy pathway proteins in intestinal immune homeostasis.

LC3-associated phagocytosis (LAP) is a non-canonical autophagy pathway that may be activated during phagocytosis upon recognition of microbes recognition receptors. Different from canonical autophagy, LAP is activated during phagocytosis upon recognition of microbes by pattern recognition receptors for rapid pathogen degradation. The efficient clearance of the infectious cargo promoted by LAP could by itself be sufficient to reduce the inflammatory response, and hence immunopathology. However, a mechanism by which inflammation is regulated during LAP has been recently described and involves the death-associated protein kinase 1 (DAPK1). LAP may be regulated by DAPK1. IFN-γ activation of DAPK1 has been shown to mediate LAP to the fungus Aspergillus fumigatus with concomitant inhibition of Nod-like receptor protein 3 (NLRP3), resulting in mitigation of pathogenic inflammation.

A granuloma is a structure formed during inflammation that is found in many diseases. It is a collection of immune cells known as macrophages. Granulomas (also referred to as granulomata) form when the immune system attempts to wall off substances it perceives as foreign but is unable to eliminate. Such substances include infectious organisms including bacteria and fungi, as well as other materials such as keratin and suture fragments. Granulomas are associated with various diseases, disorders and conditions including, but not limited to cat-scratch disease, granuloma annulare, cryptococcosis, Histoplasmosis, leprosy, Listeria monocytogenes, aspiration pneumonia, Foreign-body granuloma, childhood granulomatous periorificial dermatitis, pneumocystis pneumonia, rheumatoid arthritis, rheumatic Fever, sarcoidosis, schistosomiasis, and tuberculosis.

Chronic granulomatous disease (CGD) is a heritable immunodeficiency caused by mutations in the proteins forming the NAPDH complex that results in defective production of reactive oxygen species (ROS), impaired microbial killing by phagocytic cells and increased susceptibility to infections. This leads to the formation of painful granulomas in the affected areas. DAPK1 activity is defective in human and murine CGD. Characterization of CGD shows severe recurrent bacterial and other anti-inflammatory disorders such as non-infections severe colitis. Because although severe colitis is common in these patients and subclinical colitis is also evident in most asymptomatic patients, this suggests a dysregulated immune homeostasis at mucosal surfaces in CGD. A common feature of CGD patients is the presence of a hyperinflammatory state in multiple organs, including the gastrointestinal and urogenital tract, lungs, and eyes to which inflammation caused by defective LAP greatly contributes.

The generation of ROS by the influx of neutrophils during infection is accompanied by local oxygen consumption that results in a condition known as inflammatory hypoxia, with stabilization of the hypoxia inducible factor-1 (HIF-1)α and resolution of inflammation. This phenomenon is particularly relevant in the colonic mucosa and the effect of HIF-1α in the induction of angiogenesis- and glycolysis-related genes as well as genes involved in mucosal barrier protection has been validated in animal models of colitis and in human-derived colonic tissue. Consistent with the role of ROS in inflammatory hypoxia, the majority of CGD patients manifest inflammatory bowel disease (IBD)-like symptoms and pharmacological stabilization of HIF1α within the mucosa protected CGD mice from severe colitis.

Although the contribution of inflammatory hypoxia in the lung is disputed, hypoxia develops during pulmonary invasive fungal infection in models of invasive aspergillosis, including CGD mice, and HIF-1α stabilization is required for protection. HIF-1α mediates the autophagic process induced by a hypoxic environment. Aberrant HIF-1α induction/stabilization in CGD patients may be causally related to the impaired autophagy. Thus, pharmacological stabilization of HIF-1α might restore LAP/DAPK1 and immune homeostasis during infection in CGD.

At present the most curative treatment for patients with X-chromosome-linked CGD (X-CGD) is hematopoietic stem cell transplantation (HSCT). But for many patients without an HLA-matched donor and active infections/inflammatory complications still require novel approaches. There is a need for new treatments for granulomata and CGD.

### SUMMARY OF THE INVENTION

In one aspect, the present disclosure includes a method of treating or reducing the likelihood of the onset of an autophagy-mediated disease in a patient in need thereof, by administering to said patient a composition containing an effective amount of Thymosin β4 (Tβ4), or a fragment or isoform thereof.

In one aspect, the present disclosure includes a method of treating a subject suffering from a granuloma, by administering a composition containing an effective amount of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject.

In one aspect, the present disclosure includes a method of stabilizing hypoxia inducible factor-1 (HIF-1)α in a subject in need thereof, by administering a composition containing an effective amount of Thymosin beta 4 (β4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject, thereby stabilizing hypoxia inducible factor-1 (HIF-1)α in the subject.

In one aspect, the present disclosure includes a method of promoting autophagy in a subject in need thereof, by administering a composition containing an effective amount of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject, thereby promoting autophagy in the subject.

In one aspect, the present disclosure includes a method of upregulating genes involved in mucosal barrier protection in a subject in need thereof, by administering a composition containing an effective amount of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject, thereby upregulating genes involved in mucosal barrier protection in the subject.

In one aspect, the present disclosure includes a method of promoting LC3-associated phagocytosis in a subject in need thereof, by administering a composition containing an effective amount of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject, thereby promoting LC3-associated phagocytosis in the subject.

In one aspect, the present disclosure includes a method of promoting HIF-1α expression in a subject in need thereof, by administering a composition containing an effective amount of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject, thereby promoting HIF-1α expression in the subject.

In one aspect, the present disclosure includes a method of reducing cytokine production in a subject in need thereof, by administering a composition containing an effective amount of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject, thereby reducing cytokine production in the subject.

In one aspect, the present disclosure includes a method of promoting weight regain in a subject in need thereof, by administering a composition containing an effective amount of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject, thereby promoting weight regain in the subject.

In one aspect, the present disclosure includes a method of inhibiting granuloma formation in a subject suffering from CGD, by administering a composition containing an effective amount of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject, thereby inhibiting granuloma formation in the subject.

In one aspect, the present disclosure includes a method of increasing survival in a subject suffering from CGD, by administering a composition containing an effective amount of Thymosin beta 4 Tβ(4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject, thereby increasing survival rate in the subject.

The present inventors have discovered through in vitro and in vivo models that human and murine cells affected by CGD can be controlled by administration of Thymosin β4 (Tβ4) or variants thereof. Tβ4 is a β3 amino acid thymic hormone polypeptide providing diverse intra- and extracellular activities. The amino acid sequence of Tβ4 is disclosed in U.S. Pat. No. 4,297,276 (Goldstein), which is incorporated by reference in its entirety. Tβ4 is the major actin- sequestering molecule in all eukaryotic cells and is considered to play a significant role in the cellular metabolism due to its actin-sequestering properties. Variants of Tβ4 are disclosed throughout this disclosure for use in accordance with the disclosed treatment methods. For example, thymosin beta 4 sulfoxide is disclosed in PCT International Publication No. WO 99/49883 (Stevenson), which is incorporated by reference in its entirety. Compositions comprising oxidized or superoxidized modified normally methionine-containing beta thymosin peptides, isoforms thereof, fragments thereof, isolated R-enantiomer thereof or isolated S-enantiomer thereof, as described in U.S. Pub. No. 2008/0248993 (Hannappel), which is incorporated herein by reference in its entirety. In one aspect, peptide fragments are described in U.S. 2015/0203561 (Crockford), which is incorporated herein by reference in its entirety. In some aspects, the peptides described herein may be PEGylated. PEGylated peptides may be N-terminally PEGylated or PEGylated at various other and/or multiple positions. In some aspects, the peptides described herein may be conjugated to one or more acids, e.g., hexanoic acid and polysialic acid.

Compositions which may be used in accordance with the present invention include Thymosin β4 (Tβ4), Tβ4 isoforms, oxidized Tβ4, Thymosin (β4 sulfoxide, polypeptides or any other actin sequestering or bundling proteins having actin binding domains, or peptide fragments comprising or consisting essentially of the amino acid sequence LKKTET or conservative variants thereof. WO2000/006190 (Kleinman), incorporated herein by reference, discloses isoforms of Tβ4 which may be useful in accordance with the present invention as well as amino acid sequence LKKTET and conservative variants thereof having microbial infection-inhibiting activity, which may be utilized with the present invention. WO 99/49883 (Stevenson), incorporated herein by reference, discloses oxidized Thymosin β4 which may be utilized in accordance with the present invention. Although the present invention is described primarily hereinafter with respect to Tβ4 and Tβ4 isoforms, it is to be understood that the following description is intended to be equally applicable to amino acid sequence LKKTET, peptides and fragments comprising or consisting essentially of LKKTET, conservative variants thereof having microbial infection-inhibiting activity, as well as oxidized Thymosin β4.

Many beta thymosins and isoforms have been identified and have about 70%, or about 75%, or about 80% or more homology to the known amino acid sequence of Tβ4. Such beta thymosins and isoforms include, for example, Tβ4^{ala}, Tβ9, Tβ10, Tβ11, Tβ12, Tβ13, Tβ14 and Tβ15.

The invention is applicable to known beta thymosins, isoforms, and fragments thereof, such as those listed above, as well as normally methionine-containing beta thymosins and Tβ4 isoforms, as well as fragments thereof, identified and not yet identified. Amino acid-substituted modified beta thymosin peptides, isoforms and fragments thereof in accordance with the present invention can be provided by any suitable method, such as by solid phase peptide synthesis.

The disclosure also is applicable to methods for forming amino acid-substituted modified beta thymosin peptides, wherein the amino acid sequence of a methionine-containing beta thymosin peptide, isoform or fragment thereof is modified by substituting a non-methionine amino acid for at least one methionine in the beta thymosin peptide, isoform or fragment thereof. The method involves substituting a non-methionine amino acid for at least one methionine in a methionine-containing betathymosin peptide sequence, isoform or fragment thereof so as to form a modified beta thymosin peptide, isoform or fragment thereof

In addition, other proteins having actin sequestering or binding capability, or that can mobilize actin or modulate actin polymerization, as demonstrated in an appropriate sequestering, binding, mobilization or polymerization assay, or identified by the presence of an amino acid sequence that mediates actin binding, such as LKKTET, for example, can similarly be employed in the methods of the invention. Such proteins include gelsolin, vitamin D binding protein (DBP), profilin, cofilin, adsevertin, propomyosin, fincilin, depactin, Dnasel, vilin, fragmin, severin, capping protein, β-actinin and acumentin, for example. As such methods include those practiced in a subject, the invention further provides pharmaceutical compositions comprising gelsolin, vitamin D binding protein (DBP), profilin, cofilin, depactin, Dnasel, vilin, fragmin, severin, capping protein, β-actinin and acumentin as set forth herein. Thus, the invention includes the use of a microbial infection-inhibiting polypeptide comprising the amino acid sequence LKKTET (which may be within its primary amino acid sequence) and conservative variants thereof.

As used herein, the term “conservative variant” or grammatical variations thereof denotes the replacement of an amino acid residue by another, biologically similar residue. Examples of conservative variations include the replacement of a hydrophobic residue such as isoleucine, valine, leucine or methionine for another, the replacement of a polar residue for another, such as the substitution of arginine for lysine, glutamic for aspartic acids, or glutamine for asparagine, and the like.

The actual dosage or reagent, formulation or composition that heals damage associated with microbial infection may depend on many factors, including the size and health of a subject. However, persons of ordinary skill in the art can use teachings describing the methods and techniques for determining clinical dosages as disclosed in PCT/US99/17282, and the references cited therein, all of which are incorporated herein by reference in their entireties, to determine the appropriate dosage to use.

Compositions, as described herein, may be administered in any suitable effective amount. For example, a composition as described herein may be administered in dosages within the range of about 0.0001-1,000,000 micrograms, about 0.01-50,000 micrograms, about 0.1-10,000 micrograms, about 0.5-1,000 micrograms, about 1-500 micrograms, about 1-30 micrograms, or any amount or range within any of the recited ranges. Tβ4, or its analogues, isoforms, fragments, or variants, may be administered in any effective amount. For example, Tβ4 may be administered in dosages within the range of about 0.001-1000 micrograms of T134, about 0.01-100 micrograms, about 0.1-50, about 1-25 micrograms, or any integer or range within the disclosed ranges.

Effective dosage amounts of the Tβ4, or its analogues, isoforms, fragments, or variants may include dosage units containing about 0.01-500 mg/kg, about 1-100 mg/kg per day, about 5-50 mg/kg, about 5-20 mg/kg, about 1, 2, 3, 4, 5, 6, 7, 8, 9, or 10 mg/kg. In some aspects, dosage units are administered daily, twice per day, every other day, biweekly, or weekly.

Suitable formulations include Tβ4, a Tβ4 isoform, fragment, or variant at a concentration within the range of about 0.001-10% by weight, about 0.01-1% by weight, about 0.05-0.1% by weight, or about 0.05% by weight. In some aspects, formulations may include Tβ4, a Tβ4 isoform or variant at a concentration of about 0.0001 to 1000 mg/ml, about 0.001 to 100 mg/ml, about 0.01 to 10 mg/ml, about 0.1 to 5 mg/ml, or any concentration or concentration range within any of the recited ranges. Compositions may include Tβ4, a Tβ4 isoform or variant and a pharmaceutically acceptable carrier in a weight ratio of 1:30 to 30:1, 1:20 to 20:1, 1:10 to 10:1, or 1:15 to 1:1.

Viscosifiers may be added to adjust the viscosity of the composition and to minimize formation of impurities. Examples of viscosifiers may include polyvinyl alcohol, cellulose derivatives such as carboxymethyl cellulose and hydroxypropyl methyl cellulose and carbomer.

A composition as described herein can be administered daily, every other day, every other week, every other month, etc., with a single application or multiple applications per day of administration, such as applications 2, 3, 4 or more times per day of administration.

The therapeutic approaches described herein involve various routes of administration or delivery of reagents or compositions comprising the Tβ4 or other compounds of the invention, including any conventional administration techniques (for example, but not limited to, topical administration, local injection, inhalation, systemic or enteral administration), to a subject. The methods and compositions using or containing Tβ4 or other compounds of the invention may be formulated into pharmaceutical compositions by admixture with pharmaceutically acceptable non-toxic excipients or carriers.

The disclosure also includes a pharmaceutical composition comprising a therapeutically effective amount of a composition as described herein in a pharmaceutically acceptable carrier. Such carriers include any suitable carrier, including those listed herein.

The present disclosure shows that Tβ4 controls the LAP pathway and promotes cell autophagy by clearing the lungs of pathogens such as A. fumigatus and reducing inflammation for patients with CGD. This disclosure further shows that both autophagy and repair were dependent on HIF-1α stabilization. Accordingly, the inventors have surprisingly discovered a method of treating CGD by administering Tβ4 to a subject in need thereof. Because of the similarities between the proteins DAPK1 and Tβ4, basal autophagy is not affected by LAP, yet autophagy in response to microbial signaling is promoted by this process. Controlling autophagy in this way can benefit patients with granulomatous disorders such as CGD or schistosomiasis.

In one aspect, the present disclosure provides a method for increasing survival of a patient suffering from CGD compared to a subject not receiving the treatment. In one aspect, the survival rate is increased by at least about 10% compared to a subject not receiving the treatment. In another aspect, the survival rate is increased by at least about 20% compared to a subject not receiving the treatment. In another aspect, the survival rate is increased by at least about 30% compared to a subject not receiving the treatment. In another aspect, the survival rate is increased by at least about 40% compared to a subject not receiving the treatment. In another aspect, the survival rate is increased by at least about 50% compared to a subject not receiving the treatment. In one aspect, the survival rate is increased by about 10%-70%, including any integer or fraction thereof in the recited range, compared to a subject not receiving the treatment. In one aspect, the survival time is increased by at least 3 months compared to a subject not receiving the treatment. In one aspect, the survival time is increased by at least 6 months compared to a subject not receiving the treatment. In one aspect, the survival time is increased by at least 12 months compared to a subject not receiving the treatment. In one aspect, the survival time is increased by at least 18 months compared to a subject not receiving the treatment. In one aspect, the survival time is increased by at least 24 months compared to a subject not receiving the treatment. In one aspect, the survival time is increased by at least 36 months compared to a subject not receiving the treatment. In one aspect, the survival time is increased by at least 48 months compared to a subject not receiving the treatment. In one aspect, the survival time is increased by at least 60 months compared to a subject not receiving the treatment.

Other features and characteristics of the subject matter of this disclosure, as well as the methods of operation, functions of related elements of structure and the combination of parts, and economies of manufacture, will become more apparent upon consideration of the following description and the appended claims with reference to the accompanying drawings, all of which form a part of this specification, wherein like reference numerals designate corresponding parts in the various figures.

### BRIEF DESCRIPTION OF THE DRAWINGS

The accompanying drawings, which are incorporated herein and form part of the specification, illustrate various exemplary and non-limiting aspects of the subject matter of this disclosure.

FIG. 1A shows LC3B-II/LC3B-I expression in RAW264.7 cells after 2 or 4 hours stimulation with 10 and 100 nM T134. FIGS. 1B and 1C respectively show LC3B-II/LC3B-I and DAPK1 and Rubicon expression in RAW264.7 cells pulsed for 2 hours with A. fumigatus conidia after 1 hour pre-treatment with 10 and 100 nM of T134. In FIG. 1B, inert beads were used as control. FIG. 1D shows LC3B-II/LC3B-I and DAPK1 production in lung macrophages from C57BL/6 and p47^{phox-/-} mice pulsed with A. fumigatus conidia in the presence of Tβ4. FIG. 1E shows LC3B expression in monocytes from CGD patients or healthy control pre-treated with Tβ4 and stimulated for 2 hours with the fungus. FIGS. 1F and 1G show LC3 and DAPK1 expression on lung of C57BL/6 and p47^{phox-/-} mice infected intranasally with A. fumigatus conidia and treated intraperitoneally with 5 mg/kg Tβ4 for 7 consecutive days starting a week after the infection. FIGS. 1H and 1I show LC3-II (FIG. 1H) and DAPK1 (FIG. 1I) expression in colon lysates of C57BL/6 and p47^{phox-/-} mice subjected to DSS-induced colitis for a week and treated intraperitoneally with 5 mg/kg Tβ4 for 7 consecutive days after DSS treatment.

FIGS. 2A and 2B show Tβ4 gene expression (Ptmb4) and production in the lung of uninfected mice and Tβ4 expression in C57BL/6 and p47phox-/- mice infected intranasally with the fungus and treated with Tβ4 or siHIF-1α. FIGS. 2C and 2D show HIF-1α expression in the lung of infected mice. FIGS. 2E and 2F show HIF-1α expression in the lung of C57BL/6 and p47phox-/- mice infected and treated with Tβ4 for 7 days concomitantly to the infection. Mice were sacrificed 7 days after infection. FIGS. 2G and 2H show Tβ4 gene expression (Ptmb4) and HIF-1α gene expression in colon of mice subjected to DSS-colitis for a week and treated intraperitoneally with 5 mg/kg Tβ4 for 7 consecutive days after DSS treatment. FIG. 2I shows HIF-1α expression on monocytes from CGD patient or healthy control pre-treated with Tβ4 and stimulated for 2 hours with the fungus.

FIG. 3A shows Bnip3 and Bnip3l expression of alveolar macrophages from uninfected C57BL/6 and p47phox-/- mice pre-treated with 100 nM Tβ4 before 2 hours of pulsing with A. fumigatus conidia. FIGS. 3B and 3C show LC3 production and angiogenesis-related genes expression in C57BL/6 and p47phox-/- mice infected and treated with Tβ4 or siHIF-1α. FIG. 3D shows Expression of angiogenesis-related genes in mice subjected to DSS-induced colitis for a week and treated intraperitoneally with 5 mg/kg Tβ4 for 7 consecutive days after DSS treatment. For immunofluorescence, nuclei were counterstained with DAPI.

FIG. 4A shows lung fungal growth of C57BL/6 and p47phox-/- mice infected intranasally with A. fumigatus conidia and treated intraperitoneally with 5 mg/kg Tβ4 for 7 consecutive days starting a week after the infection. FIG. 4B shows percent of phagocytosis and conidiocidal activity on peritoneal polymorphonuclear cells and alveolar macrophages from uninfected C57BL/6 and p47phox-/- mice pre-exposed to different doses of Tβ4 for 1 hour before 2 hours of pulsing with live Aspergillus conidia. FIGS. 4C and 4D show lung gross pathology and histology (Periodic acid-Schiff staining) and NLRP3 expression of infected mice treated with Tβ4 and/or siHIF-1α. FIG. 4E shows cytokines production on lung homogenates of infected mice treated with Tβ4 or siHiflα. FIGS. 4F and 4G show T helper gene expression and cytokines production assessed in thoracic lymph nodes by real time PCR or lung homogenates by ELISA respectively.

FIGS. 5A-5G show C57BL/6 and p47phox-/- mice subjected to DSS-induced colitis for a week and treated intraperitoneally with 5 mg/kg Tβ4 1 day after DSS treatment for weight change (FIG. 5A), clinical disease activity index (FIG. 5B), histological assessment of colitis severity (H&E) (FIG. 5C), NLRP3 protein expression in colon (FIG. 5D), levels of pro-inflammatory cytokines in colon homogenates (FIGS. 5E and 5F), TGF-β production (FIG. 5G), and Cldn1 and Ocln expression in colon (FIG. 5H) .

FIGS. 6A-6E show p47phox-/- mice treated with DSS (2.5%) ad libitum in drinking water for 7 days and concomitantly treated with Tβ4 at the dose of 5 mg/kg given ip for 7 consecutive days. Seven or 14 days later, mice were evaluated for weight change (FIG. 6A), histological assessment of colitis severity (Hematoxylin and Eosin staining, 20× magnification) (FIG. 6B), Dapk1 gene expression in the colon (FIG. 6C), Cldn1 and Ocln expression (FIG. 6D) and colonic levels of cytokines (FIG. 6E).

FIGS. 7A-7D show that p47phox-/- mice with aspergillosis and treated with dimethyloxalylglycine (DMOG) for 5 days had reduced fungal burden (FIG. 7A), ameliorated lung pathology (FIG. 7B), increased HIF-1α expression (FIG. 7C) and up-regulated HIF-1α-responsive genes (FIG. 7D).

FIG. 8A shows HIF-1α-dependent glycolytic gene expression after Tβ4 treatment. FIG. 8B shows Irg1 expression in mice treated with Tβ4. FIGS. 8C and 8D show mice pre-exposed to 100 nM Tβ4 for 1 hour and pulsed with A. fumigatus conidia for additional 2 hours, in the absence (FIG. 8C) or presence (FIG. 8D) of MitoTEMPO.

FIG. 9 shows survival % of p47^{phox-/-} mice infected intranasally with A. fumigatus conidia and treated for 5 days with Tβ4 versus untreated p47^{phox-/-} mice infected intranasally with A. fumigatus conidia.

FIG. 10A shows Tβ4 (Ptmb4) and Hifla expression in the lung of C57BL/6 mice infected with A. fumigatus and treated with siTβ4, evaluated 6 days after infection. FIG. 10B shows histology (periodic acid-Schiff staining, PAS) and LC3 expression in the lung of the C57BL/6 mice infected with A. fumigatus and treated with siTβ4, evaluated 6 days after infection. Gene expression was performed by real-time PCR. Data are presented as mean ±SD and are representative of two experiments. In A−B, n=6-8 mice per group. *P<0.05, **P<0.01, siTβ4-treated vs. untreated (none) mice.

FIG. 11 schematically shows the effects of Tβ4 on tissue repair and LC3-associated phagocytosis in amelioration of disease pathologies.

### DETAILED DESCRIPTION

While aspects of the subject matter of the present disclosure may be embodied in a variety of forms, the following description and accompanying drawings are merely intended to disclose some of these forms as specific examples of the subject matter encompassed by the present disclosure. Accordingly, the subject matter of this disclosure is not intended to be limited to the forms or aspects so described and illustrated.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific aspects of the invention, but their usage does not delimit the invention, except as outlined in the claims.

As used herein, the terms “substantially” and “substantial” refer to a considerable degree or extent. When used in conjunction with, for example, an event, circumstance, characteristic, or property, the terms can refer to instances in which the event, circumstance, characteristic, or property occurs precisely as well as instances in which the event, circumstance, characteristic, or property occurs to a close approximation, such as accounting for typical tolerance levels or variability of the examples described herein.

As used herein, the term “about” is used to provide flexibility to a numerical range endpoint by providing that a given value may be “a little above” or “a little below” the endpoint. The degree of flexibility of this term can be dictated by the particular variable and would be within the knowledge of those skilled in the art to determine based on experience and the associated description herein. For example, in one aspect, the degree of flexibility can be within about ±10% of the numerical value. In another aspect, the degree of flexibility can be within about ±5% of the numerical value. In a further aspect, the degree of flexibility can be within about ±2%, ±1%, or ±0.05%, of the numerical value.

Generally herein, the term “or” includes “and/or.”

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilizing (i.e. not worsening) the state of disease, delaying or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. In addition to being useful as methods of treatment, the methods described herein may be useful for the prevention or prophylaxis of disease. As used herein, the term “treating” refers to any administration of a compound of the present invention and includes (i) inhibiting the disease in an animal that is experiencing or displaying the pathology or symptomatology of the diseased (i.e., arresting further development of the pathology and/or symptomatology) or (ii) ameliorating the disease in an animal that is experiencing or displaying the pathology or symptomatology of the diseased (i.e., reversing the pathology and/or symptomatology). The term “controlling” includes preventing treating, eradicating, ameliorating or otherwise reducing the severity of the condition being controlled.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0” should be interpreted to include not only the explicitly recited values of about 0.01 to about 2.0, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. Furthermore, such an interpretation should apply regardless of the breadth of the range or the characteristics being described. Additionally, it is noted that all percentages are in weight, unless specified otherwise.

In understanding the scope of the present disclosure, the terms “including” or “comprising” and their derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps. It is understood that reference to any one of these transition terms (i.e. “comprising,” “consisting,” or “consisting essentially”) provides direct support for replacement to any of the other transition term not specifically used. For example, amending a term from “comprising” to “consisting essentially of” would find direct support due to this definition.

As used herein, a plurality of compounds or steps may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

The term “compound” or “agent”, as used herein, unless otherwise indicated, refers to any specific peptide, fragment or isoform disclosed herein.

The terms “administration of” or “administering a” compound as used herein should be understood to mean providing a compound of the invention to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as intravenous (IV), bolus injection, intramuscular (IM), intraperitoneal (IP), intranasal, and the like; enteral or parenteral, transdermal dosage forms, including creams, jellies, powders, or patches; buccal dosage forms; inhalation powders, sprays, suspensions, and the like; and rectal suppositories.

Depending upon the particular route of administration desired a variety of pharmaceutically acceptable carriers well known in the art may be used. These include solid or liquid fillers, diluents, hydrotropes, surface-active agents, and encapsulating substances. Optional pharmaceutically active materials may be included, which do not substantially interfere with the activity of the one or more active agents.

As used herein the term “intravenous administration” includes injection and other modes of intravenous administration.

The term “pharmaceutically acceptable” as used herein to describe a carrier, diluent or excipient must be compatible with the other ingredients of the formulation and not deleterious to the recipient thereof. The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation.

Thus, another aspect of the present invention provides pharmaceutically acceptable compositions comprising an effective amount of one or more agents, formulated together with one or more pharmaceutically acceptable carriers (additives) and/or diluents. As described below, the pharmaceutical compositions of the present invention may be specially formulated for administration in solid or liquid form, including those adapted for the following: (1) local administration to the central nervous system, for example, intrathecal, intraventricular, intraspinal, or intracerebrospinal administration (2) oral administration, for example, drenches (aqueous or non-aqueous solutions or suspensions), tablets, boluses, powders, granules, pastes for application to the tongue; (3) parenteral administration, for example, by subcutaneous, intramuscular or intravenous injection as, for example, a sterile solution or suspension; (4) topical application, for example, as a cream, ointment or spray applied to the skin; or (5) ophthalmic administration, for example, for administration following injury or damage to the retina. However, in certain embodiments the subject agents may be simply dissolved or suspended in sterile water. In certain embodiments, the pharmaceutical preparation is non-pyrogenic, i.e., does not elevate the body temperature of a patient.

Some examples of the pharmaceutically acceptable carrier materials that may be used include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain aspects, the thymosin-β4 polypeptides or variants of the present disclosure may contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts the thymosin-β4 polypeptide. These salts can be prepared in situ during the final isolation and purification of the agents of the invention, or by separately reacting a purified agent of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like. (See, for example, Berge et al. (1977) “Pharmaceutical Salts”, J. Pharm. Sci. 66:1-190.). The pharmaceutically acceptable salts include the conventional nontoxic salts or quaternary ammonium salts of the agents, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the agent which produces a therapeutic effect. Generally, out of one hundred percent, this amount could range from about 1 percent to about ninety-nine percent of active ingredient, or from about 5 percent to about 70 percent, or even from about 10 percent to about 30 percent.

Methods of preparing these formulations or compositions include the step of bringing into association an agent with the carrier and, optionally, one or more accessory ingredients. In general, the formulations are prepared by uniformly and intimately bringing into association an agent of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a agent of the present invention as an active ingredient. An agent of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration of the agents of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof

Besides inert diluents, the oral compositions can also include adjuvants such as wetting agents, emulsifying and suspending agents, sweetening, flavoring, coloring, perfuming and preservative agents.

Suspensions, in addition to the active agents, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Transdermal patches have the added advantage of providing controlled delivery of an agent of the present invention to the body. Such dosage forms can be made by dissolving or dispersing the agents in the proper medium. Absorption enhancers can also be used to increase the flux of the agents across the skin. The rate of such flux can be controlled by either providing a rate controlling membrane or dispersing the agent in a polymer matrix or gel.

Pharmaceutical compositions of this invention suitable for parenteral administration comprise one or more agents of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of an agent, it is desirable to slow the absorption of the agent from subcutaneous or intramuscular injection. This may be accomplished by the use of a liquid suspension of crystalline or amorphous material having poor water solubility. The rate of absorption of the agent then depends upon its rate of dissolution which, in turn, may depend upon crystal size and crystalline form. Alternatively, delayed absorption of a parenterally administered agent form is accomplished by dissolving or suspending the agent in an oil vehicle.

Another aspect of the present invention provides a packaged pharmaceutical. In one embodiment, the packaged pharmaceutical comprises (i) a thymosin-β4 polypeptide, or functional variant thereof, in therapeutically effective amounts and in a ready-to-use dosage form; and (ii) instructions and/or a label for administration of the therapeutic agents for the treatment of subjects having an autophagy-mediated condition, e.g., CGD.

The term “patient” or “subject” is used throughout the specification within context to describe an animal, generally a mammal, including a domesticated mammal including a farm animal (dog, cat, horse, cow, pig, sheep, goat, etc.) or a human, to whom treatment, including prophylactic treatment (prophylaxis), with the methods and compositions according to the present invention is provided. For treatment of those conditions or disease states which are specific for a specific animal such as a human patient, the term patient refers to that specific animal, often a human.

The terms “effective” or “pharmaceutically effective” are used herein, unless otherwise indicated, to describe an amount of a compound or composition which, in context, is used to produce or affect an intended result, usually treatment of a disease, disorder or condition within the context of a particular treatment or alternatively, the effect of a peptide, protein, fragment, or isoform of the present disclosure, which is co-administered with another autophagy modulator and/or another bioactive agent in the treatment of disease.

The terms “treat”, “treating”, and “treatment”, etc., as used herein, refer to any action providing a benefit to a patient at risk for or afflicted by an autophagy mediated disease state or condition as otherwise described herein. The benefit may be in curing the disease state or condition, inhibition its progression, or ameliorating, lessening or suppressing one or more symptom of an autophagy mediated disease state or condition. Treatment, as used herein, encompasses both prophylactic and therapeutic treatment.

A method of treatment of a subject in need thereof according to certain aspects of the invention, comprises at least one of:

treating a subject suffering from a granuloma comprising administering a composition comprising an effective amount of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject;

stabilizing hypoxia inducible factor-1 (HIF-1)α in a subject in need thereof, comprising administering a composition comprising an effective amount of Thymosin beta 4 (Tβ4), a T(β4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject, thereby stabilizing hypoxia inducible factor-1 (HIF-1)α in the subject;

promoting autophagy in a subject in need thereof, comprising administering a composition comprising an effective amount of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject, thereby promoting autophagy in the subject;

upregulating genes involved in mucosal barrier protection in a subject in need thereof, comprising administering a composition comprising an effective amount of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject, thereby upregulating genes involved in mucosal barrier protection in the subject;

promoting LC3-associated phagocytosis in a subject in need thereof, comprising administering a composition comprising an effective amount of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject, thereby promoting LC3-associated phagocytosis in the subject;

promoting HIF-1α expression in a subject in need thereof, comprising administering a composition comprising an effective amount of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject, thereby promoting HIF-1α expression in the subject;

reducing cytokine production in a subject in need thereof, comprising administering a composition comprising an effective amount of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject, thereby reducing cytokine production in the subject;

promoting weight regain in a subject in need thereof, comprising administering a composition comprising an effective amount of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject, thereby promoting weight regain in the subject;

inhibiting granuloma formation in a subject suffering from CGD comprising administering a composition comprising an effective amount of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject, thereby inhibiting granuloma formation in the subject; or

increasing survival in a subject suffering from CGD comprising administering a composition containing an effective amount of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject, thereby increasing survival rate in the subject.

According to certain aspects, the before mentioned cytokine is at least one of IL-1β, IL-17A, TNF-α, and IFN-γ.

According to certain aspects, the subject suffers from chronic granulomatous disease (CGD).

According to certain aspects, the composition is administered systemically, nasally, orally, or intravenously.

According to certain aspects, the composition is suitable for topical delivery, inhalation, systemic administration, oral administration, intranasal administration, intravenous administration, intraperitoneal administration, intramuscular administration, intracavity administration or transdermal administration. According to certain aspects, the Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof is recombinant or synthetic.

According to certain aspects, a Tβ4 isoform is Tβ4^{ala}, Tβ9, Tβ10, Tβ11, Tβ12, Tβ13, Tβ14 or Tβ15. According to certain aspects, a composition comprises about 0.1-50 micrograms of Thymosin beta 4 (Tβ34), a Tβ34 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof.

According to certain aspects, about 0.01-500 mg/kg of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variant thereof is administered to the subject.

According to certain aspects, a composition contains about 0.001-10% by weight of the Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variant thereof.

According to certain aspects, a composition is administered daily, twice per day, every other day, biweekly, or weekly. According to certain aspects, a composition contains the Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variant thereof at a ratio of 1:30 to 30:1 to the pharmaceutically acceptable carrier.

According to certain methods of the present disclosure, administration of Tβ4 exerts effective therapeutic activity by promoting autophagy. This effect is demonstrated in in vitro and in vivo models, e.g., as understood when cells affected with CGD are exposed to irritants of dextran sodium sulfate (DSS) and microbial stimuli A. fumigatus. This mechanism involves the activation of DAPK1 through LAP. As disclosed herein, experimental studies in vitro and in vivo with human cells and mice with CGD unexpectedly demonstrated that LAP using DAPK1 is promoted by Tβ4 when in the presence of A. fumigatus. Tests were done using mice with CGD to monitor inflammation in both the lungs and intestines. Using murine cells in vitro, DAPK1 and Tβ4 were both defective in those affected by CGD. The data show that Tβ4 balances inflammation and growth of granulomas as seen in cases of non-infectious granulomatous disorders, e.g., due to the increase in activity of phagocytes to stop the growth of granulomas. The method of the present disclosure is useful for effecting a matrix protein synthesis process that repairs granuloma healing mechanisms with help of TGF-β when stimulated appropriately by Tβ4.

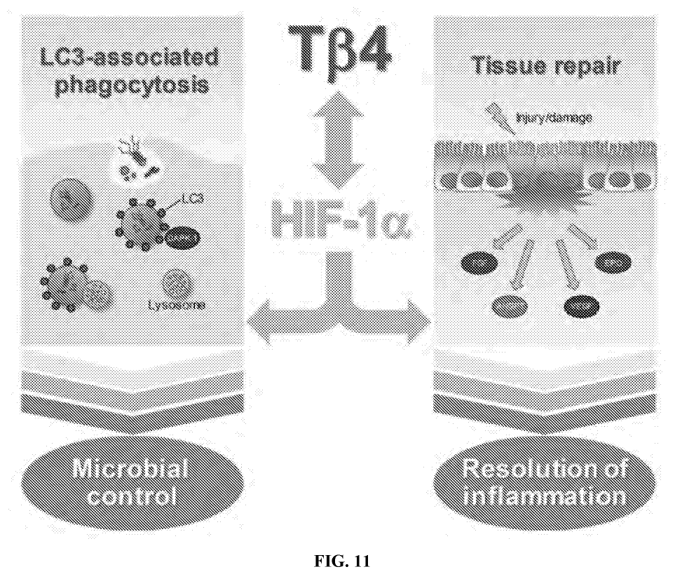

As shown in FIG. 11, Tβ4 restores autophagy and upregulates hypoxi-responsive genes in human and murine CGD, resulting in amelioration of disease pathology.

### EXAMPLES

Aspects of the present disclosure will be further described with reference to the following Examples, which are provided for illustrative purposes only and should not be used to limit the scope of or construe the invention.

### Example 1

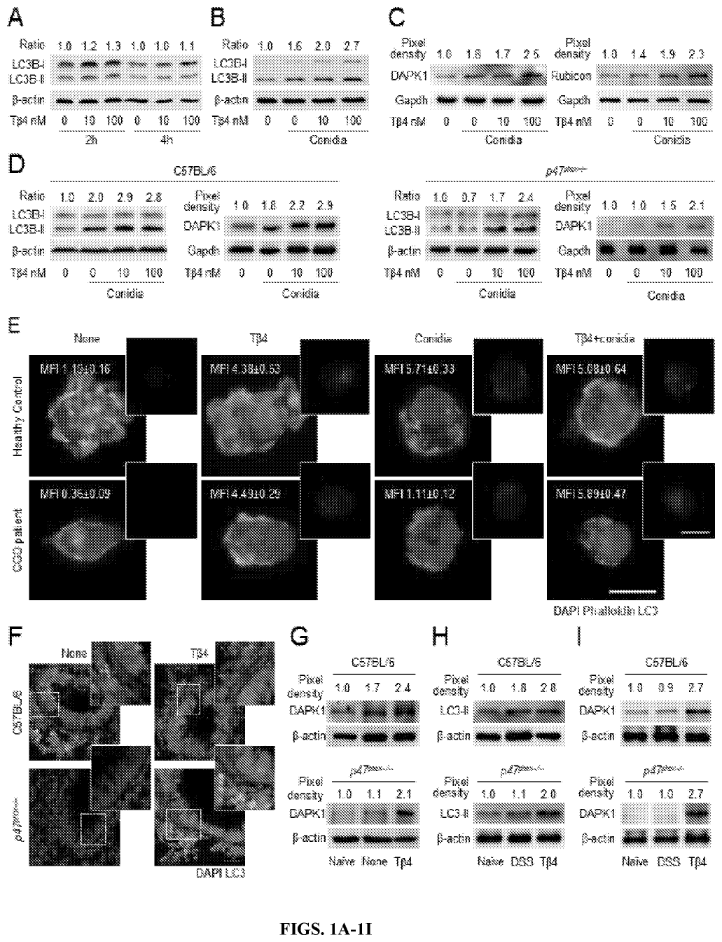

The ability of Tβ4 to promote autophagy was assessed by the determining the ratio of LC3-II to LC3-I, widely used to monitor autophagy (Oikonomou et al., 2016) on RAW 264.7 cells exposed to live Aspergillus conidia in the presence of different concentrations of Tβ4. Tβ4 did not induce autophagy in unpulsed cells (FIG. 1A), but dose-dependently increased the LC3-II to LC3-I ratio in cells pulsed with conidia. This effect was observed as early as 2 hours after the exposure to the fungus (FIG. 1B). This finding suggests that Tβ4 could be able to activate LAP. Accordingly, the experiment testing the effect of Tβ4 on DAPK1 and Rubicon proteins, shows the expression of DAPK1 and Rubicon proteins, known to be involved in LAP, were dose-dependently increased by Tβ4 (FIG. 1C). As a result, Tβ4 may promote non-canonical autophagy involving DAPK1 and Rubicon.

Next, macrophages were purified from lungs of C57BL/6 and p47phox-/- mice and pulsed in vitro with A. fumigatus conidia in the presence of Tβ4. Both autophagy and DAPK1 expression were defective in cells from p47phox-/- mice but were dose-dependently restored by Tβ4 (FIG. 1D). Tβ4 also increased LC3B expression in monocytes from CGD patients exposed to Aspergillus conidia in vitro (FIG. 1E). Therefore, the data suggest that Tβ4 can restore LAP involving DAPK1 in human CGD.

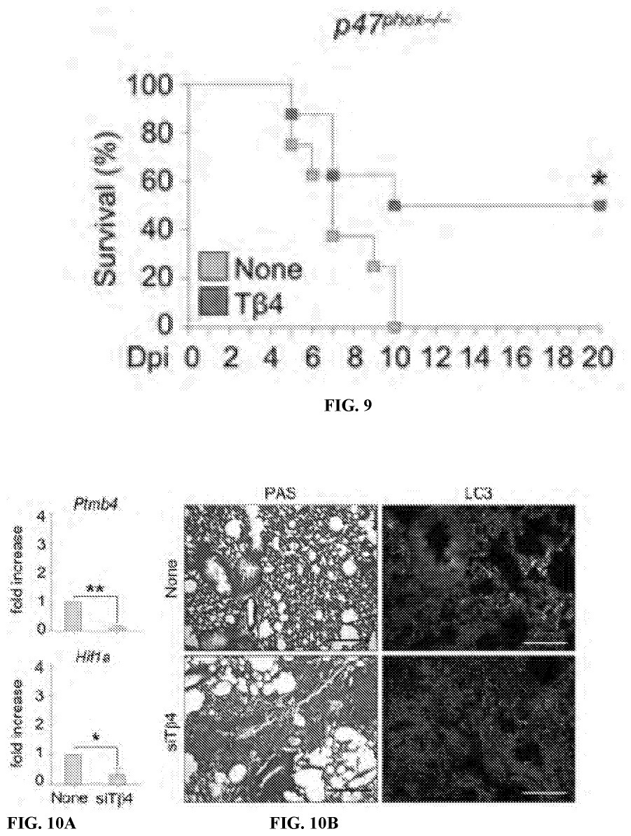

In vivo models mimicking human pathology such as lung and gut inflammation were prepared. A model of lung inflammation was tested using C57BL/6 and p47phox-/- mice. The mice were infected with A. fumigatus intranasally and treated them with Tβ4 for 7 consecutive days starting a week after the infection. LC3-II (FIG. 1F) and DAPK1 (FIG. 1G) expression were both defective in p47phox-/- mice, but restored by Tβ4 and ablated (LC3-II) upon siTβ4 (FIGS. 10A and 10B). In addition, gut inflammation was tested using the same mouse model. Acute colitis in p47phox-/- mice was initiated by administering 2.5 DSS in drinking water for 7 days followed by 7 days of DSS-free autoclaved water. Subsequently, Tβ4 was therapeutically administered daily for 7 days, after DSS treatment, at the time at which mice started to lose weight. Consistent with previous findings (de Luca et al., 2014), LC3-II (FIG. 1H) and DAPK1 (FIG. 1I) expression were both defective in the colon ofp47phox-/- mice with colitis as opposed to WT mice but restored upon treatment with T134 (FIGS. 1H and 1I). DAPK1 expression in colon lysates of C57BL/6 and p47^{phox-/-} mice subjected to DSS-induced colitis for a week and treated intraperitoneally with 5 mg/kg Tβ4 for 7 consecutive days after DSS treatment, n=6-10 mice per group. Data are presented as mean ±SD and are representative of two experiments. Mice were categorized as Naive (uninfected or untreated mice) or None (infected mice). For immunoblotting, normalization was performed on mouse β-actin or Gapdh and corresponding pixel density or ratio is depicted. For immunofluorescence, nuclei were counterstained with DAPI. Photographs were taken with a high-resolution microscope (Olympus BX51), 40× and 100× magnification. LC3 mean fluorescence intensity (MFI) was measured with the ImageJ software. In FIGS. 1F-1H, n=6-10 mice per group. Data are presented as mean ±SD and are representative of two experiments. Naïve, uninfected or untreated mice. None, infected mice.

### Example 2

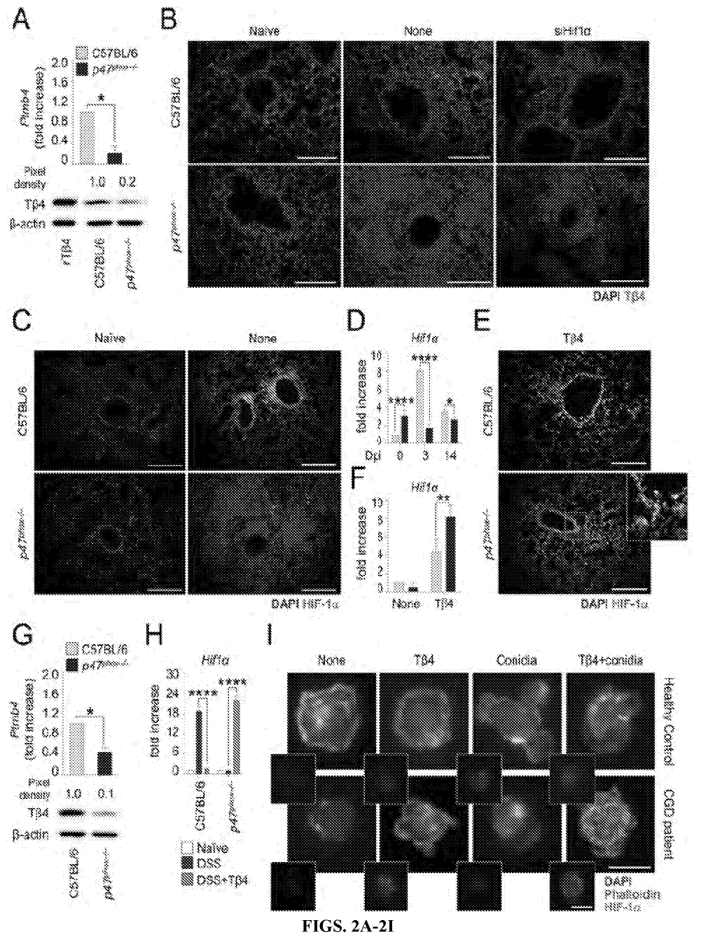

The inventors of the present disclosure provide an unexpected method for treating CGD by administering an effective amount of Tβ4, e.g., by administering a sufficient amount to promote HIF-1α expression in a subject suffering from CGD. Given that the defective LAP in CGD is amenable to restoration by Tβ4, experiments were conducted to determine whether the production of Tβ4 is defective in CGD by assessing Tβ4 gene and protein expression in p47phox-/- mice. A lower expression was observed for Tβ4 in CGD mice as compared to C57BL/6 mice, both in terms of gene and protein expression, in the lungs (FIG. 2A-2B) and colons (FIG. 2F).

Given the reciprocal regulation between Tβ4 and HIF-1α, an experiment was conducted to determine whether defective Tβ4 levels in CGD mice could be associated with altered HIF-1α expression. HIF-1α gene and protein expression was measured in a p47phox-/- CGD mouse model. HIF-1α protein levels were reduced in CGD mice (FIGS. 2C and 2D).

Next, an experiment was conducted to show that the administration of exogenous Tβ4 restored HIF-1α protein in CGD mice (FIG. 2E and 2F), whereas HIF-1α silencing decreased Tβ4 expression in C57BL/6 mice (FIG. 2B). Defective Tβ4 expression (FIG. 2G) and restoration of HIF-1α expression upon administration of Tβ4 (FIG. 2H) was also observed in the colon. Consistent with the murine results, Tβ4 also increased HIF-1α expression in monocytes from CGD patients challenged with Aspergillus conidia (FIG. 2I), thus suggesting that Tβ4 is able to restore HIF-1α expression in human CGD. Immunoblotting, normalization was performed on mouse β-actin or GAPDH and corresponding pixel density or ratio is depicted. For immunofluorescence, nuclei were counterstained with DAPI. Photographs were taken with a high-resolution microscope (Olympus BX51), 40× and 100× magnification. LC3 mean fluorescence intensity (MFI) was measured with the ImageJ software. *p<0.05, p47^{phox-/-} vs C57BL/6 mice. None, unpulsed and untreated cells. Ctrl, Recombinant (r) Tβ4 used as positive control. These results show that intracellular autocrine crosstalk between Tβ4 expression and HIF-1α induction occurs in CGD.

### Example 3

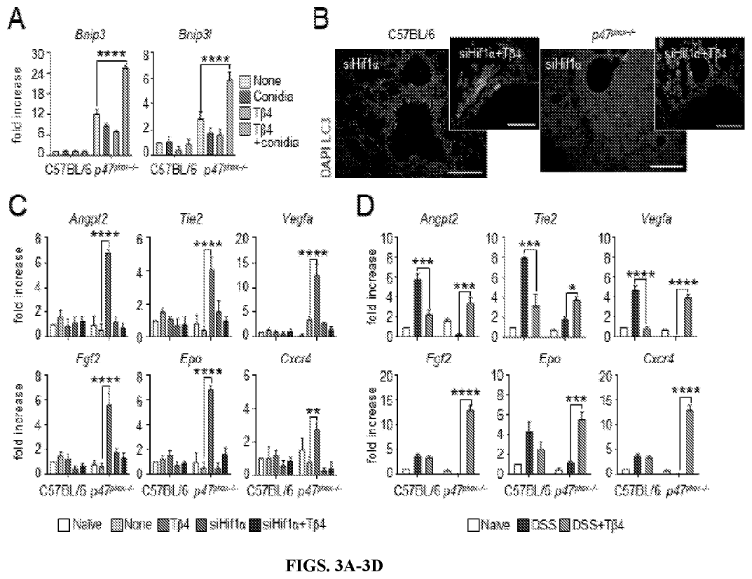

The inventors of the present disclosure found that Tβ4 promotes LAP and mucosal barrier protection in an HIF-1α dependent manner. Experiments were conducted to determine whether a causal link exists between HIF-1α stabilization and induction of autophagy by Tβ4. A gene expression experiment showed that Tβ4 induced the expression in vitro of Bnip3 and Bnip3l, which are known to be involved in hypoxia-induced autophagy (FIG. 3A). A further experiment was conducted to infect p47phox-/- mice with A. fumigatus intranasally. The mice were treated with Tβ4 in the presence or absence of a siRNA for HIF-1α. HIF-1α inhibition abrogated LC3-II expression in p47phox-/- mice by Tβ4 (FIG. 3B). This experiment shows that Tβ4 requires HIF-1α to induce LAP.

Next, experiments were conducted to determine whether Tβ4 is involved in mucosal protection. Specifically, an experiment was conducted to measure in vivo gene expression following Tβ4 treatment. Genes involved in angiogenic signaling (Angpt2, Tie2, Vegfa), remodeling (Fgf2), hormonal regulation (Epo), and cell migration (Cxcr4) were found to be upregulated in the lungs of Aspergillus-infected mice upon treatment with Tβ4; treatment with HIF-1α siRNA completely abrogated the upregulation induced by Tβ4 (FIG. 3C). The results show that the gene expression is HIF-1α mediated. A similar experiment using colon homogenates of p47phox-/- mice in the DSS-induced colitis model showed upregulation of these genes by Tβ4 (FIG. 3D). These results show that HIF-1α mediates fundamental effects of Tβ4, including LAP and induction of genes involved in the angiogenesis and repair. Accordingly, based on the present disclosure, a person skilled in the art would readily appreciate that the Tβ4-HIF-1α axis is a potential therapeutic pathway in CGD.

### Example 4

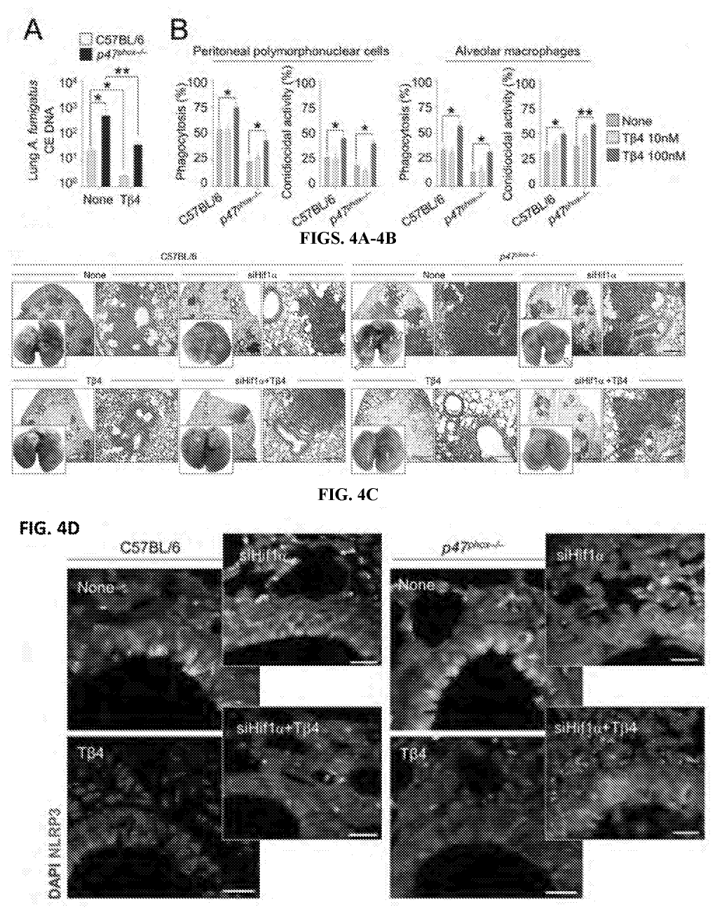

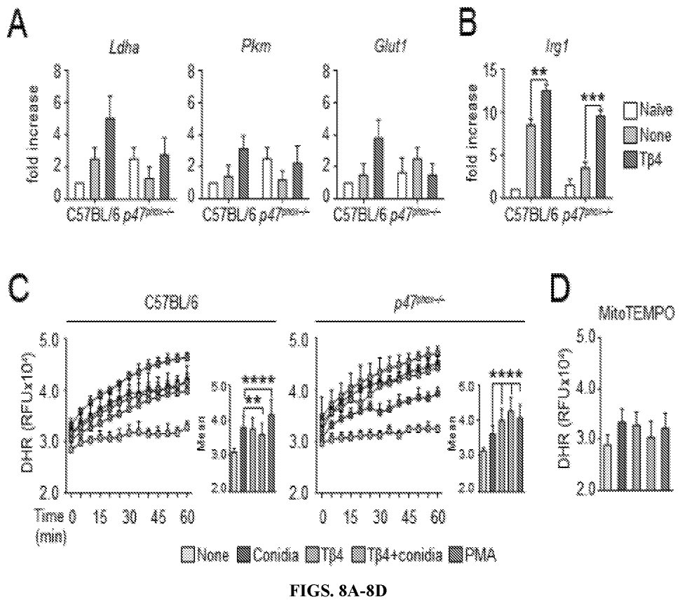

To determine whether Tβ4 ameliorates tissue and immune pathologies in CGD mice the inventors of the present disclosure conducted experiments measuring the effect of Tβ4 in mice with aspergillosis and colitis. Specifically, mice were monitored for fungal growth, antifungal activity of effector cells, survival, lung histopathology, innate and adaptive Th immunity. In this experiment, Tβ4 reduced the fungal growth in the lung of both types of mice (FIG. 4A), an effect to which the ability of T134 to potentiate phagocytosis and fungal killing of effector phagocytes likely contributed (FIG. 4B), and significantly increased the survival of infected mice in that more than 50% of mice survived at the time when all untreated mice had died (FIG. 9). Gross lung pathology and histological examination was conducted on p47phox-/- mice. No signs of inflammatory lung injury and granuloma formation after Tβ4 administration was observed (FIG. 4C). Conversely, Tβ4 deficiency by means of siTβ4 administration promoted lung pathology in C57BL/6 mice (FIG. 10B). Tβ4 treatment down-regulated NLRP3 expression in an experiment conducted with these mice (FIG. 4D) and, accordingly, reduced IL-1β, along with TNF-α, IL-17A and IFNy production, an effect negated upon siHifla treatment (FIG. 4E).

Further, an experiment an experiment was conducted to measure the effect of Tβ4 treatment on pathogenic and protective cell responses. Following Tβ4 treatment, pathogenic Th2/Th17/Th9 cell responses became down-regulated and protective Th1/Treg cell responses were promoted (FIG. 4C and 4D). An experiment using HIF-1α siRNA showed that the above effects on tissue pathology were all abolished when HIF-1α is silenced (FIG. 4C). This experiment shows the importance of HIF-1α in mediating Tβ4 effects. For immunofluorescence, nuclei were counterstained with DAPI. Photographs were taken with a high-resolution microscope (Olympus BX51), 4×, 20× and 40× magnification. Secreted cytokines were assayed by ELISA from supernatants. Data are presented as mean ±SD and are representative of two experiments. In A-E, n=6 mice per group. *P<0.05, **P<0.01, ***P<0.001, p47phox-/- vs C57BL/6 mice, Tβ4-treated vs untreated (None) mice or cells. None, scrambled mice. Naïve, uninfected mice.

### Example 5

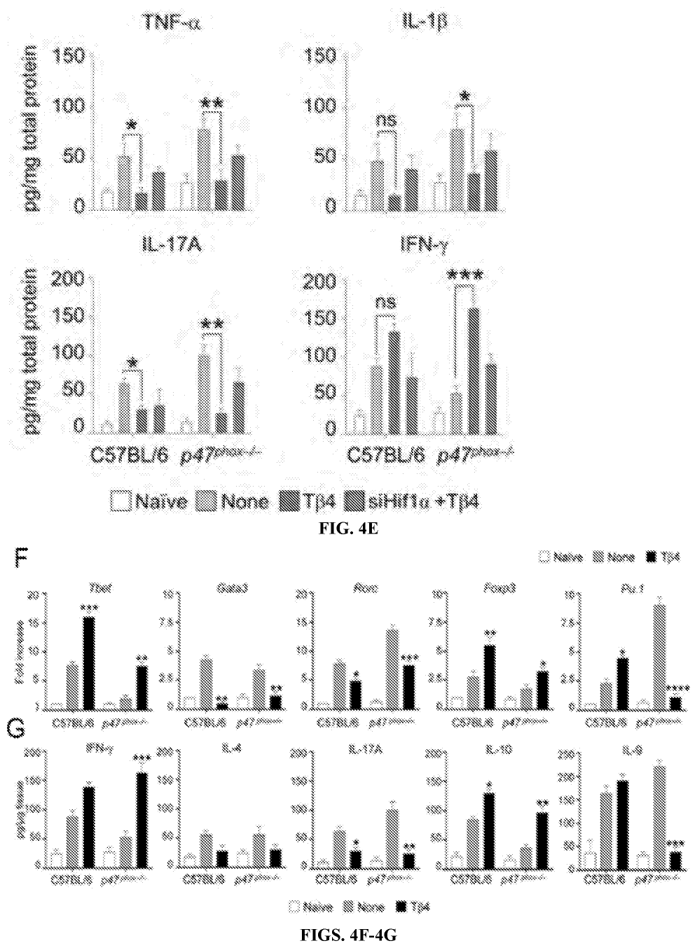

In experiments using a murine colitis model, mice were evaluated a day after Tβ4 treatment for weight loss, colon histology, cytokine levels and tight junction gene expression. p47phox-/- mice lost more weight than C57BL/6 mice (about 50% loss of their initial body weight on day 14) (FIG. 5A) and had more severe colitis as evidenced by significantly increased disease activity index scores (FIG. 5B). Hematoxylin and eosin (H&E) staining of colon sections showed severe patchy inflammation characterized by transmural lymphocytic infiltrates, epithelial ulceration, and complete crypt loss (FIG. 5C). In addition, p47phox-/- mice displayed high levels of NLRP3 expression (FIG. 5D), and IL-1β production (FIG. 5E) along with high levels of myeloperoxidase (MPO), TNF-α and IL-17A (FIG. 5F) and low levels of TGF-β (FIG. G). In contrast, treatment with Tβ4 significantly led to weight regain (FIG. 5A), a decreased disease activity index scores (FIG. 5B), amelioration of inflammatory pathology and tissue architecture (FIG. 5C), decreased NLRP3 expression (FIG. 5D) and inflammatory cytokine levels (FIGS. 5E and 5F) and up-regulation of the anti-inflammatory cytokines (FIG. 5G). Tβ4 greatly promoted the expression of both Cldn1 and Ocln, tight junction proteins that regulate intestinal permeability (FIG. 5H). This suggests a positive effect on the mucosal barrier function.

### Example 6

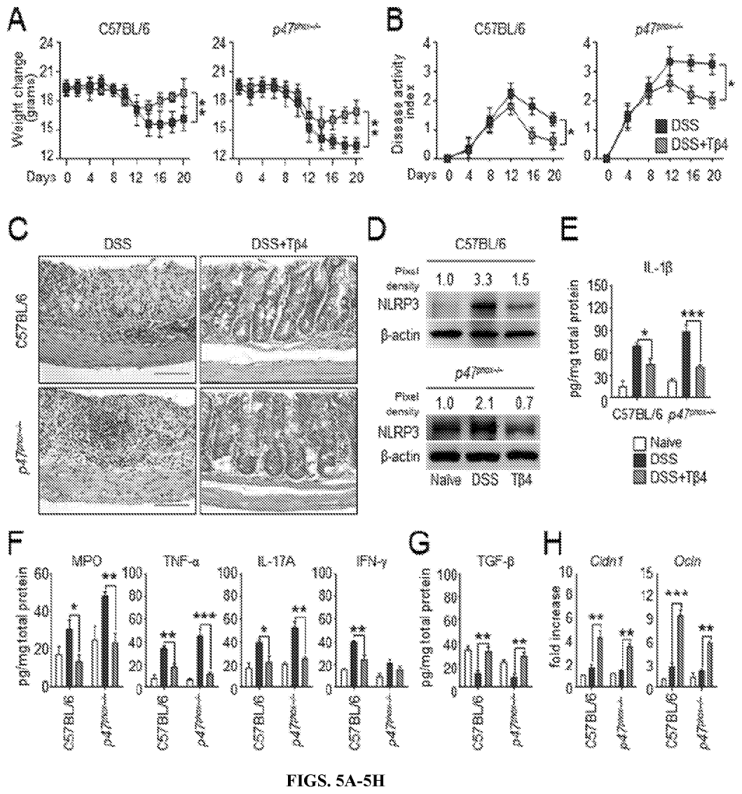

An experiment was conducted to test the protective effect of Tβ4 when treatment was given concomitantly with DSS. Specifically, p47phox-/- mice received DSS (2.5%) ad libitum in drinking water for 7 days. Tβ4 at the dose of 5 mg/kg was given ip for 7 consecutive days concomitantly with the DSS treatment (FIG. 6). Seven and 14 days later, mice were evaluated for weight change (FIG. 6A), histological assessment of colitis severity (FIG. 6B), Dapk 1 gene expression in the colon (FIG. 6C), Cldn1 and Ocln expression (FIG. 6D) and colonic levels of cytokines (FIG. 6E). Photographs were taken with a high-resolution microscope (Olympus BX51), 20× magnification. Secreted cytokines were assayed by ELISA from supernatants. Gene expression was performed by real time-PCR. Data are presented as mean ±SD and are representative of two experiments. In A-E, n=10 mice per group. *P<0.05, **P<0.01, ***P<0.001, Tβ4-treated vs untreated (DSS) mice.-These results show that Tβ4, by activating LAP-DAPK1 and inhibiting inflammasome activity, could have beneficial effects on the outcome of colitis in CGD.

### Example 7

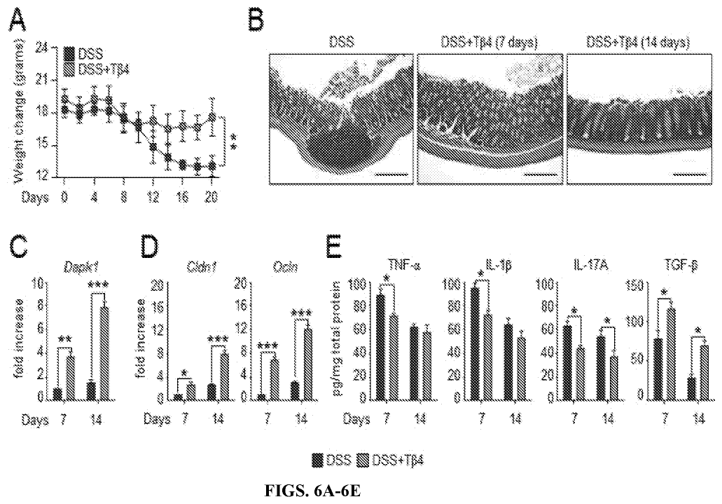

Experiments were conducted to determine whether HIF-1α stabilization recapitulates the effects of Tβ4. p47phox-/- aspergillosis mice were treated dimethyloxalylglycine (DMOG)—a cell permeable competitive inhibitor of prolyl hydroxylase (PHD) that stabilizes HIF-1α—for 5 days. DMOG reduced fungal burden (FIG. 7A), ameliorated lung pathology (FIG. 7B), increased HIF-1α expression (FIG. 7C) and up-regulated HIF-1α-responsive genes (FIG. 7D). This experiment shows that HIF-1α stabilization could be a therapeutic target in CGD.

### Example 8

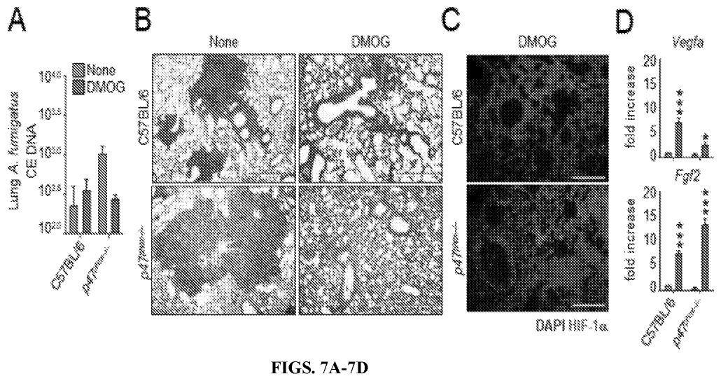

Experiments were also conducted to determine whether Tβ4 increases mitochondrial redox balance. p47phox-/- mice were infected intranasally with A. fumigatus and treated with 5 mg/kg Tβ4. HIF-1α-dependent glycolytic genes were not increased by Tβ4 treatment (FIG. 8A). In contrast, Irg1 expression was increased in mice treated with Tβ4 (FIG. 8B). In addition, ROS production by DHR was measured in C57BL/6 and p47phox-/- mice pre-exposed to 100 nM T134 for 1 hour and pulsed with A. fumigatus conidia for additional 2 hours, in the absence (FIG. 8C) or presence (FIG. 8D) of MitoTEMPO. We could detect the production of mitochondrial ROS induced by Tβ4, thus supporting the hypothesis that mtROS might mediate the regulation of HIF-1α levels by Tβ4. 10 ng/ml PMA was used as a positive control. Fluorescence was measured by a Tecan Infinite 200 fluorimeter. Data are presented as mean ±SD and are representative of two independent experiments. In A-B, n=6 mice per group. **P <0.01, ***P<0.001, ****P<0.0001, Tβ4-treated vs untreated (None) mice, treated vs untreated (Conidia) cells. RFU, relative fluorescence units.

The present disclosure surprisingly shows that the effects of Tβ4 are dependent on HIF-1α. These effects mediate not only the induction of autophagy but also the upregulation of hypoxic-responsive genes. The present disclosure shows the unique ability of administering Tβ4 to a subject in need thereof to activate physiologic HIF-1α to resolve inflammation. Elevation of HIF-1α levels restores hypoxia-mediated tissue homeostasis as well as the optimal anti-microbial response. Further, experiments of the present disclosure show the biological activity of Tβ4 in CGD, illuminating the importance of mtROS production and HIF-1α stabilization as druggable pathways promoting autophagy and repair in CGD.

### Materials and Methods Used in Examples

RAW264.7 cells (ATCC) were grown in supplemented RPMI medium. Cells were exposed to 10 or 100 nM of Tβ4 (RegeneRx Biopharmaceuticals, Rockville, Md., USA) for 2 and 4 hours at 37° C. in 5% CO2 or pre-treated for 1 hour with Tβ4 at the same concentration before 2 hours pulsing with live A. fumigatus conidia or inert beads (LB30, Sigma Aldrich). Alveolar macrophages from lung of C57BL/6 and p47phox-/- uninfected mice were obtained after 2 hours of plastic adherence at 37° C. Cells were treated as above and evaluated for cellular autophagy markers. Monocytes were isolated from PBMC of healthy donors or two CGD patients, harboring the mutations c.736C>T, p.Q246X and whole CYBB gene deletion (69,84 kb), followed informed consent, as described (De Luca et al., 2012 CD4(+) T cell vaccination overcomes defective cross-presentation of fungal antigens in a mouse model of chronic granulomatous disease. J Clin Invest 122:1816-1831). Cells were assessed for LC3 and HIF-1α expression by immunofluorescence.

Viable conidia from the A. fumigatus Af293 strain were obtained as described (De Luca et al., 2012). Mice were anesthetized in a plastic cage by inhalation of 3% isoflurane (Forane Abbot) in oxygen before intranasal instillation of 2×10⁷ resting conidia/20 μl saline. For survival curves, p47^{phox-/-} mice were challenged with 3×10⁹ conidia/20 μl saline. Tβ4 was administered intraperitoneally (i.p.) at the dose of 5 mg/kg at an effective dose as described (Badamchian et al., Thymosin beta(4) reduces lethality and down-regulates inflammatory mediators in endotoxin-induced septic shock. Int Immunopharmacol, 2003 3(8): p. 1225-33), every day in concomitance with (days 0→7) or after (days 7→14) infection. DMOG (Merck Millipore) was administered i.p. at the dose of 8 mg/mouse concomitantly to the infection. For Hif1a silencing, each mouse received intranasal administration of 10 mg/kg unmodified siRNA [Duplex name mm.Ri.Hif1a.13.1 (IDT); 5′-GAUAUGUUUACUAAAGGACAAGUCA-3′; 3′-UACUAUACAAAUGAUUUCCUGUUCAGU -5′] and Tmsb4x silencing [Duplex name mm.Ri.Tmsb4x.13.1; 5′-CACAUCAAAGAAUCAGAACUACUGA-3′; 3′-AAGUGUAGUUUCUUAGUCUUGAUGACU -5′], or equivalent dose of nonspecific control siRNA duplex in a volume of 20 μl of duplex buffer (IDT). Intranasal siRNA was given once the day before infection and 1, 3 and 5 days after infection (Iannitti et al., 2013 Hypoxia promotes danger-mediated inflammation via receptor for advanced glycation end products in cystic fibrosis. Am J Respir Crit Care Med 188:1338-1350). It is known that lung-specific siRNA delivery can be achieved by intranasal administration without the use of viral vectors or transfection agents in vivo (Iannitti et al., 2013 Th17/Treg imbalance in murine cystic fibrosis is linked to indoleamine 2,3-dioxygenase deficiency but corrected by kynurenines. Am J Respir Crit Care Med 187:609-620). Mice were sacrificed 7 or 14 days post infection. Fungal burden was determined by qPCR and expressed as conidial equivalents (CE). Lung tissue was aseptically removed and homogenized in 3 ml of sterile saline. Lung homogenates were subjected to a secondary homogenization step with 0.5 mm glass beads in Bead Beater homogenizer (Gemini BV) and then processed for DNA extraction with the QlAamp DNA Mini Kit (Qiagen) according to the manufacturer's directions. Fungal burden was quantified by qPCR by using the sequences for the multicopy 18S ribosomal DNA gene. For lung histology, sections (3-4 μm) of paraffin-embedded tissues were stained with Periodic acid-Schiff (PAS).

Dextran Sulfate Sodium (DSS) (2.5% wt/vol, 36,000-50,000 kDa; MP Biomedicals) was administered in drinking water ad libitum for 7 days. Fresh solution was replaced on day 3. Mice were injected with 5 mg/kg of Tβ4 every day intraperitoneally in concomitance with (days 0→7) or after (days 7→14) DSS administration. Control received the diluent alone. Weight loss, stool consistency, and faecal blood were recorded daily. Upon necropsy (7 and 14 days after DSS administration), tissues were collected for histology and cytokine analysis. Colonic sections were stained with Hematoxylin and Eosin. Colitis disease activity index was calculated daily for each mouse based on weight loss, occult blood, and stool consistency. A score of 1-4 was given for each parameter as described (McNamee et al., 2011 Interleukin 37 expression protects mice from colitis. Proc Natl Acad Sci U S A 108:16711-16716).

For immunoblotting, organs or cells were lysed in RIPA buffer. The lysate was separated in sodium dodecyl sulfate-polyacrylamide gel electrophoresis (SDS-PAGE) and transferred to a nitrocellulose membrane. The membranes were incubated with the following primary antibodies at 4oC overnight: anti-DAPK1 (antibodies-online.com), anti-Rubicon (Cell Signaling), anti-Tβ4 and anti-NLRP3 (Abcam), anti-LC3B (Cell Signaling or Abcam). Normalization was performed by probing the membranes with mouse anti-β-actin and anti-Gapdh antibodies (Sigma-Aldrich). Normalization was performed on mouse β-actin or Gapdh and corresponding pixel density is depicted. LC3-II band density was normalized to LC3-I to obtain ratio. Chemiluminescence detection was performed with LiteAblot Plus chemiluminescence substrate (EuroClone S.p.A.), using the ChemiDocTM XRS+ Imaging System (Bio-Rad) and quantification was obtained by densitometry image analysis using Image Lab 5.1 software (Bio-Rad).

For immunofluorescence, monocytes from CGD patients or controls were grown in supplemented RPMI and placed on microscope glass slides at 37° C. for adhesion. Slides were then washed with PBS and fixed with 4% of paraformaldehyde. Cells were incubated in blocking solution (PBS-3% BSA-0.1%-Triton X-100) with anti-LC3B antibody (Nanotools) and anti-HIF-1α (Abcam). After overnight staining with primary antibodies, slides were washed and incubated with anti-IgG and rabbit-TRITC (Sigma Aldrich). Alexa Fluor® 488 phalloidin was used for selective labelling of F-actin. LC3B (Abcam), Tβ4 (ABclonal), HIF-1α and NLRP3 (Abcam) staining of lung sections were done as described. Nuclei were counterstained with DAPI. Images were acquired using a fluorescence microscope (BX51, Olympus) and the analySIS image processing software (Olympus).

Real-time RT-PCR was performed using CFX96 Touch Real-Time PCR Detection System and SYBR Green chemistry (Bio-Rad). Organs or cells were lysed and total RNA was reverse transcribed with PrimeScript RT Reagent Kit with gDNA Eraser (Takara), according to the manufacturer's instructions. The PCR primers sequences (5′-3′) were as follows:

Ptmb4:
ACAAACCCGATATGGCTGAG
and
GCCAGCTTGCTTCTCTTGTT
Hif1a:
TCAAGTCAGCAACGTGGAAG
and
TTCACAAATCAGCACCAAGC
Hif1b:
CAAGCATCTTTCCTCACTGATC
and
ACACCACCCGTCCAGTCTCA
Cldn1:
AGCCAGGAGCCTCGCCCCGCAG
CTGCA
and
CGGGTTGCCTGCAAAGT
Ocln:
GTTGATCCCCAGGAGGCTAT
and
CCATCTTTCTTCGGGTTTTC
Vegfa:
CAGGCTGCTGTAACGATGAA
and
GCATTCACATCTGCTGTGCT
Fgf2:
CGACCCACACGTCAAACTAC
and
GCCGTCCATCTTCCTTCATA
Bnip3:
GCTCCCAGACACCACAAGAT
and
TGAGAGTAGCTGTGCGCTTC
Bnip3l:
CCTCGTCTTCCATCCACAAT
and
GTCCCTGCTGGTATGCATCT
Angpt2:
GAACCAGACAGCAGCACAAA
and
TGGTCTGATCCAAAATCTGCT
Tie2:
CGGCCAGGTACATAGGAGGAA
and
TCACATCTCCGAACAATCAGC
Epo:
ACTCTCCTTGCTACTGATTCCT
and
ATCGTGACATTTTCTGCCTCC
Cxcr4:
GGGTCATCAAGCAAGGATGT
and
GGCAGAGCTTTTGAACTTGG
Dapk1:
CCTGGGTCTTGAGGCAGATA
and
TCGCTAATGTTTCTTGCTTGG
Ldha:
AGGCTCCCCAGAACAAGATT
and
TCTCGCCCTTGAGTTTGTCT
Pktn:
CGATCTGTGGAGATGCTGAA
and
AATGGGATCAGATGCAAAGC
Glut1:
GCTGTGCTTATGGGCTTCTC
and
CACATACATGGGCACAAAGC
Irg1:
AGTTCCAACACCTCCAGCAC
and
GGTGCCATGTGTCATCAAAA

Amplification efficiencies were validated and normalized against Gapdh. The thermal profile for SYBR Green real-time PCR was at 95° C. for 3 min, followed by 40 cycles of denaturation for 30 sec at 95° C. and an annealing/extension step of 30 sec at 60° C. Each data point was examined for integrity by analysis of the amplification plot.

To evaluate cytokine production in DSS colitis, colons were opened longitudinally and washed in complete medium with antibiotics, then were cultured at 37° C. for 24 hours in RPMI and 5% FBS. The supernatants were collected for ELISA. The levels of cytokines were determined by specific ELISAs (R&D System) in accordance with the manufacturer's protocols. The concentration of secreted cytokines in the colon supernatants or lung homogenates was normalized to total tissue protein by using Quant-iT Protein Assay Kit (Life Technologies). Results are expressed as picogram of cytokine per microgram of total protein. Myeloperoxidase (MPO) content in colonic tissues were determined using commercially available kits (Nanjing Jiancheng Bioengineering Institute).

Murine polymorphonuclear cells (PMNs) from C57BL/6 or p47phox-/- uninfected mice were positively selected with magnetic beads (Miltenyi Biotech) from the peritoneal cavity of mice 8 hours after the intraperitoneal injection of 1 ml endotoxin-free 10% thioglycolate solution. On fluorescence-activated cell sorting (FACS) analysis, Gr-1+PMNs were 98% pure and stained positive for the CD11b myeloid marker. Monolayers of plastic-adherent macrophages were obtained, after 2 hours plastic adherence, from lung of C57BL/6 and p47phox-/- uninfected mice. Cells were pre-treated for 1 hour with different concentrations of Tβ4 (10 and 100 nM) before pulsing with A. fumigatus conidia (1:3 cell/fungus ratio for phagocytosis and 10:1 cell/fungus for conidiocidal activity) for 120 min at 37° C. The percentage of CFU inhibition (mean±SD) was determined as described previously (Bellocchio et al., 2004 TLRs govern neutrophil activity in aspergillosis. J Immunol 173:7406-7415).

Alveolar macrophages from lung of C57BL/6 and p47phox-/- uninfected mice were assessed for intracellular ROS production by dihydrorhodamine 123 (DHR) evaluation. The MitoTEMPO inhibitor was used to scavenge mitochondrial ROS. For ROS determination, 10 μM DHR (Sigma-Aldrich) were added to cells exposed to 100 nM T134, 10 ng/ml PMA (phorbol 12-myristate 13-acetate) (Sigma-Aldrich) and/or A. fumigatus conidia at cells:fungi ratio of 1:1 for 1 hour at 37° C. Cells were plated on a 96 wells culture plate in HBSS buffer with Ca2+and Mg230 but without phenol red. Cells were pre-incubated with 50 μM MitoTEMPO (Enzo Life Science) for 1 hour before the addition of Tβ4. The DHR was measured by the multifunctional microplate reader Tecan Infinite 200 (Tecan) at different time points. The results expressed as relative fluorescence units (RFU) are the means ±SD of at least two experiments in duplicate.

GraphPad Prism 6.01 program (GraphPad Software) was used for analysis. Data are expressed as mean ±SD. Statistical significance was calculated by two-way ANOVA (Tukey's or Bonferroni's post hoc test) for multiple comparisons. Statistical analysis of the survival curves was performed using the Log-rank (Mantel-Cox) test. The data reported are either from one representative (histology, immunofluorescence and western blotting) or pooled otherwise. The in vivo groups consisted of 6-10 mice/group. The variance was similar in the groups being compared. Cell fluorescence intensity was measured by using the ImageJ software.

While the subject matter of this disclosure has been described and shown in considerable detail with reference to certain illustrative examples, including various combinations and sub-combinations of features, those skilled in the art will readily appreciate other aspects and variations and modifications thereof as encompassed within the scope of the present disclosure. Moreover, the descriptions of such aspects, combinations, and sub-combinations is not intended to convey that the claimed subject matter requires features or combinations of features other than those expressly recited in the claims. Accordingly, the scope of this disclosure is intended to include all modifications and variations encompassed within the spirit and scope of the following appended claims.

1. A method of treatment of a subject in need thereof, comprising at least one of:
A) treating a subject suffering from a granuloma comprising administering a composition comprising an effective amount of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject;
B) stabilizing hypoxia inducible factor-1 (HIF-1)α in a subject in need thereof, comprising administering a composition comprising an effective amount of Thymosin beta 4 (Tβ4), a Tβ4 isoform, oxidized Tββ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject, thereby stabilizing hypoxia inducible factor-1 (HIF-1)α in the subject;
C) promoting autophagy in a subject in need thereof, comprising administering a composition comprising an effective amount of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject, thereby promoting autophagy in the subject;
D) upregulating genes involved in mucosal barrier protection in a subject in need thereof, comprising administering a composition comprising an effective amount of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject, thereby upregulating genes involved in mucosal barrier protection in the subject;
E) promoting LC3-associated phagocytosis in a subject in need thereof, comprising administering a composition comprising an effective amount of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject, thereby promoting LC3-associated phagocytosis in the subject;
F) promoting HIF-1α expression in a subject in need thereof, comprising administering a composition comprising an effective amount of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject, thereby promoting HIF-1α expression in the subject;
G) reducing cytokine production in a subject in need thereof, comprising administering a composition comprising an effective amount of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject, thereby reducing cytokine production in the subject;
H) promoting weight regain in a subject in need thereof, comprising administering a composition comprising an effective amount of Thymosin beta 4 Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject, thereby promoting weight regain in the subject;
I) inhibiting granuloma formation in a subject suffering from CGD comprising administering a composition comprising an effective amount of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject, thereby inhibiting granuloma formation in the subject; or
J) increasing survival in a subject suffering from CGD comprising administering a composition containing an effective amount of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof and a pharmaceutically acceptable carrier to the subject, thereby increasing survival rate in the subject.

2. The method of claim 1, wherein the cytokine is at least one of IL-1β, IL-17A, TNF-α, and IFN-γ.

3. The method of claim 1, wherein said subject suffers from chronic granulomatous disease (CGD).

4. The method of claim 1, wherein said composition is administered systemically.

5. The method of claim 1, wherein said composition is administered nasally.

6. The method of claim 1, wherein said composition is administered orally.

7. The method of claim 1, wherein said composition is administered intravenously.

8. The method of claim 1, wherein the composition is suitable for topical delivery, inhalation, systemic administration, oral administration, intranasal administration, intravenous administration, intraperitoneal administration, intramuscular administration, intracavity administration or transdermal administration.

9. The method of claim 1, wherein the Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof is recombinant or synthetic.

10. The method of claim 1, wherein the Tβ4 isoform is Tβ4^{ala}, Tβ9, Tβ10, Tβ11, Tβ12, Tβ13, Tβ14 or Tβ15.

11. The method of claim 1, wherein the composition comprises about 0.1-50 micrograms of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variants thereof.

12. The method of claim 1, wherein about 0.01-500 mg/kg of Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variant thereof is administered to the subject.

13. The method of claim 1, wherein the composition contains about 0.001-10% by weight of the Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variant thereof

14. The method of claim 1, wherein the composition is administered daily, twice per day, every other day, biweekly, or weekly.

15. The method of claim 1, wherein the composition contains the Thymosin beta 4 (Tβ4), a Tβ4 isoform, oxidized Tβ4, Thymosin β4 sulfoxide, a polypeptide or any other actin sequestering or bundling proteins having an actin binding domain, or a peptide fragment comprising amino acid sequence LKKTET or conservative variant thereof at a ratio of 1:30 to 30:1 to the pharmaceutically acceptable carrier.
